(19) United States
(12) Patent Application Publication
(10) Pub. No.: US 2025/0070457 A1
(43) Pub. Date: Feb. 27, 2025

(54) SMART CONTACT LENS

(71) Applicant: OPTHROTH INC., Gyeonggi-do (KR)

(72) Inventors: SI YOUL YOO, Gyeonggi-do (KR); HAE YOUNG KIM, Seoul (KR); HYO JIN JEONG, Gyeonggi-do (KR)

(21) Appl. No.: 18/720,849

(22) PCT Filed: Dec. 7, 2022
(86) PCT No.: PCT/KR2022/019822

§ 371 Date: Jun. 17, 2024

(30) Foreign Application Priority Data
Dec. 17, 2021 (KR) 10-2021-0182195
Aug. 25, 2022 (KR) 10-2022-0106980

(51) Int. Cl. H01Q 1/27; A61B 3/16; A61B 5/00; G02C 7/04; H02J 50/20

(52) U.S. Cl. CPC H01Q 1/273; A61B 5/6821; G02C 7/04; A61B 3/16; A61B 2562/0261; H02J 50/20

(57) ABSTRACT

A smart contact lens includes a lens body including a first lens surface in contact with an eye and a second lens surface positioned opposite the first lens surface, and an antenna disposed between the first lens surface and the second lens surface and configured to form a closed loop. The antenna may include an antenna ring forming a shape of a ring as a whole; and an antenna buffer bent and extended from the antenna ring in a diameter direction of the antenna ring.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2022/019822 filed on Dec. 7, 2022, which claims priority to the benefit of Korean Patent Application Nos. 10-2021-0182195 filed on Dec. 17, 2021 and 10-2022-0106980 filed on Aug. 25, 2022 in the Korean Intellectual Property Office, the entire contents of which are incorporated herein by reference.

### BACKGROUND

### 1. Technical Field

The present disclosure relates to a smart contact lens. More particularly, the present disclosure relates to a smart contact lens that effectively embeds an antenna including a planar shape in the contact lens.

### 2. Background Art

Research on smart devices is very active as smart wearable devices that are made small and light to be worn on the body and improve convenience.

In keeping with the development of e-health systems, numerous research companies around the world are developing various electronic devices to diagnose and treat human diseases, and a particularly useful application area is ophthalmic lenses. For example, wearable lenses may include a lens assembly with electronically adjustable focus to improve eye performance. For another example, wearable contact lenses with or without adjustable focus may include an electronic sensor to detect concentrations of specific chemicals in the precorneal membrane. The use of embedded electronic devices in the lens assembly introduces potential requirements for how to communicate with and supply power and re-energy to the electronic devices.

Embedding the electronic devices and communication capability in the contact lenses requires technical challenges in a number of areas, including limited size of components, particularly, a thickness as well as a maximum length and width, limited energy storage capacity in batteries or supercapacitors, limited peak current consumption due to high battery internal resistance in small batteries, limited charge storage in small capacitors, limited average power consumption due to limited energy storage, and limited robustness and manufacturability of small and especially thin components.

### SUMMARY

An object of the present disclosure is to address the above-described and other problems.

Another object of the present disclosure is to provide a smart contact lens including an antenna that minimizes corrugations and folds of the antenna in a process of making the antenna on a plane in a three-dimensional shape.

In order to achieve the above-described and other objects and needs, in one aspect of the present disclosure, there may be provided a smart contact lens comprising a lens body including a first lens surface in contact with an eye and a second lens surface positioned opposite the first lens surface; and an antenna disposed between the first lens surface and the second lens surface and configured to form a closed loop, the antenna including an antenna ring forming a shape of a ring as a whole; and an antenna buffer bent and extended from the antenna ring in a diameter direction of the antenna ring.

Effects of a smart contact lens according to the present disclosure are described as follows.

According to at least one aspect of the present disclosure, there can be provided a smart contact lens including an antenna that minimizes corrugations and folds of the antenna in a process of making the antenna on a plane in a three-dimensional shape.

Additional scope of applicability of the present disclosure will become apparent from the detailed description given blow. However, it should be understood that the detailed description and specific examples such as embodiments of the present disclosure are given merely by way of example, since various changes and modifications within the spirit and scope of the present disclosure will become apparent to those skilled in the art from the detailed description.

### BRIEF DESCRIPTION OF THE DRAWINGS

FIG. 1 is a perspective view of a smart contact lens according to an embodiment of the present disclosure.

FIG. 2 is a plan view of the smart contact lens illustrated in FIG. 1.

FIG. 3 illustrates a cross section of the smart contact lens taken along line A-A of FIG. 2.

FIG. 4 illustrates an antenna formed on a plane.

FIG. 5 illustrates a cross section of the antenna taken along line B-B of FIG. 4.

FIG. 6 illustrates a contrast between a planar antenna and a three-dimensional antenna.

FIG. 7 illustrates a planar antenna provided with an antenna inner buffer.

FIG. 8 illustrates a planar antenna provided with an antenna outer buffer.

FIG. 9 illustrates a planar antenna provided with an antenna outer buffer and an antenna inner buffer.

FIGS. 10 to 17 illustrate a planar antenna according to various embodiments of the present disclosure.

### DETAILED DESCRIPTION

Reference will now be made in detail to embodiments of the disclosure, examples of which are illustrated in the accompanying drawings. Wherever possible, the same reference numbers will be used throughout the drawings to refer to the same or like parts. In general, a suffix such as “module” and “unit” may be used to refer to elements or components. Use of such a suffix herein is merely intended to facilitate description of the present disclosure, and the suffix itself is not intended to give any special meaning or function. It will be noted that a detailed description of known arts will be omitted if it is determined that the detailed description of the known arts can obscure embodiments of the present disclosure. The accompanying drawings are used to help easily understand various technical features and it should be understood that embodiments presented herein are not limited by the accompanying drawings. As such, the present disclosure should be construed to extend to any alterations, equivalents and substitutes in addition to those which are particularly set out in the accompanying drawings.

The terms including an ordinal number such as first, second, etc. may be used to describe various components, but the components are not limited by such terms. The terms are used only for the purpose of distinguishing one component from other components.

When any component is described as “being connected” or “being coupled” to other component, this should be understood to mean that another component may exist between them, although any component may be directly connected or coupled to the other component. In contrast, when any component is described as “being directly connected” or “being directly coupled” to other component, this should be understood to mean that no component exists between them.

A singular expression can include a plural expression as long as it does not have an apparently different meaning in context.

In the present disclosure, terms “include” and “have” should be understood to be intended to designate that illustrated features, numbers, steps, operations, components, parts or combinations thereof are present and not to preclude the existence of one or more different features, numbers, steps, operations, components, parts or combinations thereof, or the possibility of the addition thereof.

In the drawings, sizes of the components may be exaggerated or reduced for convenience of explanation. For example, the size and the thickness of each component illustrated in the drawings are arbitrarily illustrated for convenience of explanation, and thus the present disclosure is not limited thereto unless specified as such.

If any embodiment is implementable differently, a specific order of processes may be performed differently from the order described. For example, two consecutively described processes may be performed substantially at the same time, or performed in the order opposite to the described order.

In the following embodiments, when layers, areas, components, etc. are connected, the following embodiments include both the case where layers, areas, and components are directly connected, and the case where layers, areas, and components are indirectly connected to other layers, areas, and components intervening between them. For example, when layers, areas, components, etc. are electrically connected, the present disclosure includes both the case where layers, areas, and components are directly electrically connected, and the case where layers, areas, and components are indirectly electrically connected to other layers, areas, and components intervening between them.

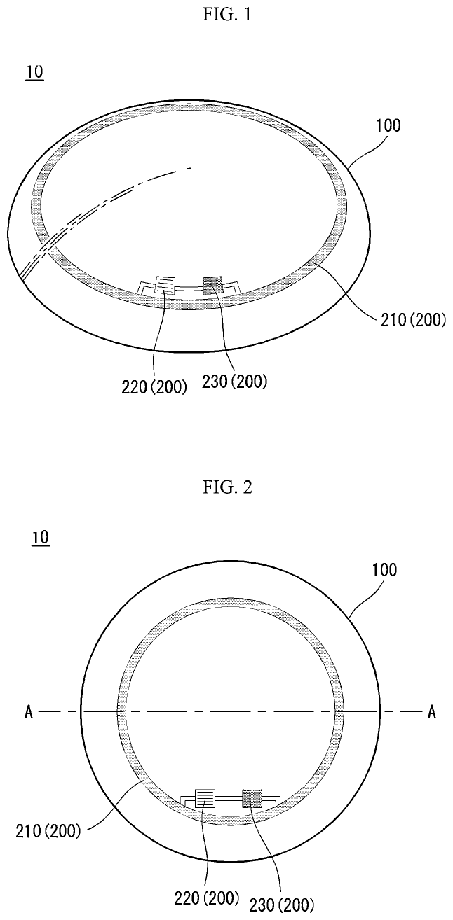

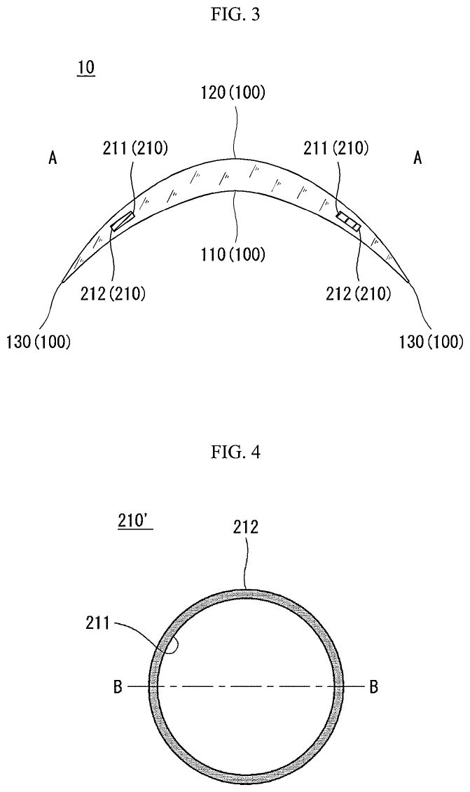

FIG. 1 is a perspective view of a smart contact lens according to an embodiment of the present disclosure. FIG. 2 is a plan view of the smart contact lens illustrated in FIG. 1. FIG. 3 illustrates a cross section of the smart contact lens taken along line A-A of FIG. 2.

Referring to FIGS. 1 to 3, a smart contact lens 10 includes a lens body 100. The lens body 100 may form a skeleton of the smart contact lens 10. The lens body 100 may form an exterior surface of the smart contact lens 10.

The lens body 100 may be formed of a material containing a transparent material. For example, the lens body 100 may be formed of a material containing silicon. For example, the lens body 100 may be formed of a material that combines silicone, a thermal initiator, a UV blocker, hydroxyethyl methacrylate (HEMA), etc.

The lens body 100 may include a first lens surface 110. The first lens surface 110 may be a surface formed on the lens body 100. The first lens surface 110 may face the eye and be in contact with the eye. A shape of the first lens surface 110 may correspond to a shape of the eye.

The lens body 100 may include a second lens surface 120. The second lens surface 120 may be another surface formed on the lens body 100. The second lens surface 120 may be positioned opposite the first lens surface 110.

The lens body 100 may include a lens outer periphery 130. The lens outer periphery 130 may form borders of the first lens surface 110 and the second lens surface 120. The lens outer periphery 130 may be connected to the first lens surface 110 and the second lens surface 120.

A thickness of the lens body 100 may increase from the lens outer periphery 130 to the center of the lens body 100. The thickness of the lens body 100 may be a distance between the first lens surface 110 and the second lens surface 120. For example, the thickness of the lens body 100 may indicate a length of the lens body 100 in a direction perpendicular to the first lens surface 110 and/or the second lens surface 120.

The smart contact lens 10 may include an electronic module 200. The electronic module 200 may be embedded in the lens body 100. For example, the electronic module 200 may be disposed between the first lens surface 110 and the second lens surface 120.

The electronic module 200 may include an antenna 210. The antenna 210 may form a ring shape. The antenna 210 may form a loop. For example, the antenna 210 may form a closed loop. The antenna 210 may transmit and receive signals. The antenna 210 may receive power wirelessly from the outside.

The antenna 210 may be formed of a material containing metal. For example, the antenna 210 may be formed of a material containing at least one of gold (Ag) and copper (Cu). For example, the antenna 210 may be formed of a metal strip. A thickness of the antenna 210 may be less than the thickness of the lens body 100 at a point where the antenna 210 is positioned.

The antenna 210 may form a width. An antenna inner side 211 may indicate an inner perimeter of the antenna 210. An antenna outer side 212 may indicate an outer perimeter of the antenna 210. The width of the antenna 210 may indicate a distance between the antenna inner side 211 and the antenna outer side 212.

The electronic module 200 may include a sensor 220. The sensor 220 may include a strain sensor. For example, when the smart contact lens 10 is worn on the eye, the sensor 220 may detect expansion and contraction of the eye and/or changes in intraocular pressure. The sensor 220 may be made of a material containing a transparent material.

The sensor 220 may be connected to the antenna 210. Information acquired by the sensor 220 may be sensed by an external device through the antenna 210 or transmitted to the external device through the antenna 210. The information acquired by the sensor 220 may be biometric information. Information on the expansion and contraction of the eye and/or the changes in the intraocular pressure may be one of the biometric information. The sensor 220 may receive power through the antenna 210.

The electronic module 200 may include a chip 230. The chip 230 may include an application-specific integrated circuit (ASIC) chip. The chip 230 may generate signals. For example, the chip 230 may receive information (or signals) acquired from the sensor 220 and generate a new signal.

The chip 230 may be connected to the antenna 210. The chip 230 may receive power from the outside through the antenna 210. For example, the antenna 210 may receive power from the outside through electromagnetic induction. The antenna 210 may transfer power to the chip 230.

The chip 230 may transmit the signals to the outside. For example, the chip 230 may transmit a signal including the biometric information to the external device through wireless communication. For example, the chip 230 may include an RF communication module to transmit the signals to the external device. For another example, the chip 230 may transmit the signals to the external device through the antenna 210.

The first lens surface 110 may be concave toward a direction in which the first lens surface 110 faces. The second lens surface 120 may be concave toward a direction in which the first lens surface 110 faces. The width of the antenna 210 may be greater than the thickness of the lens body 100 at the point where the antenna 210 is positioned. Thus, the antenna 210 may form a shape corresponding to the shape of the lens body 100. For example, the shape of the antenna 210 may correspond to a portion of a cone. In other words, the antenna 210 may not be positioned on a plane.

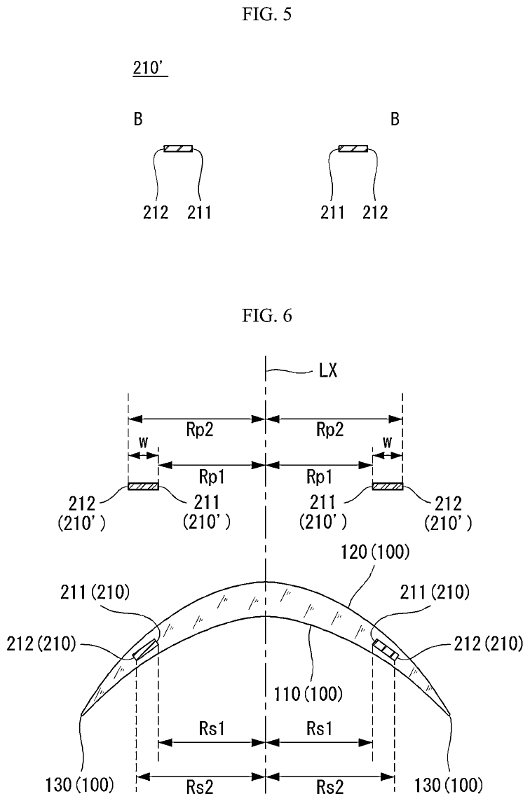

FIG. 4 illustrates an antenna formed on a plane. FIG. 5 illustrates a cross section of the antenna taken along line B-B of FIG. 4.

Referring to FIGS. 4 and 5, an antenna 210′ may be formed on a plane. For example, the antenna inner side 211 and the antenna outer side 212 may be positioned on the same plane. The cost and process time for manufacturing the antenna 210′ when the antenna 210′ is formed on the plane may be respectively less than the cost and process time for manufacturing the antenna 210 (see FIGS. 1 to 3) when the antenna 210 (see FIGS. 1 to 3) is formed in a three-dimensional shape.

Referring to FIGS. 1 to 5, in order for the antenna 210 to be embedded in the lens body 100, the antenna 210′ formed on the plane needs to be converted into a three-dimensional shape. In order to convert the antenna 210′ of a plane shape into a three-dimensional shape, pressure may be applied to the antenna 210′ of the plane shape in a mold of a desired three-dimensional shape. The antenna 210′ of the plane shape may be referred to as a “planar antenna.” The antenna 210 of the three-dimensional shape may be referred to as a “three-dimensional antenna.”

FIG. 6 illustrates a contrast between a planar antenna and a three-dimensional antenna.

Referring to FIG. 6, the planar antenna 210′ may be provided with pressure by a mold. The centers of the planar antenna 210′ and the three-dimensional antenna 210 may be positioned on a reference line LX. The planar antenna 210′ and the three-dimensional antenna 210 may be formed or arranged symmetrically with respect to the reference line LX.

Inner radii of the antennas 210 and 210′ may be specified by the antenna inner side 211. Outer radii of the antennas 210 and 210′ may be specified by the antenna outer side 212.

A planar antenna inner radius Rp1 may be an inner radius of the antenna 210′ in the planar antenna 210′. A planar antenna outer radius Rp2 may be an outer radius of the antenna 210′ in the planar antenna 210′. An antenna width w in the planar antenna 210′ may be a value by subtracting the planar antenna inner radius Rp1 from the planar antenna outer radius Rp2.

A three-dimensional antenna inner radius Rs1 may be an inner radius of the antenna 210 in the three-dimensional antenna 210. A three-dimensional antenna outer radius Rs2 may be an outer radius of the antenna 210 in the three-dimensional antenna 210. An antenna width w of the three-dimensional antenna 210 may be the same as the antenna width w of the planar antenna 210′.

A process of converting the planar antenna 210′ into the three-dimensional antenna 210 will be described.

It may be assumed that the outer radii of the antennas 210′ and 210 do not change. That is, it may be assumed that the three-dimensional antenna outer radius Rs2 is the same as the planar antenna outer radius Rp2. Assuming that the three-dimensional antenna outer radius Rs2 is the same as the planar antenna outer radius Rp2, the three-dimensional antenna inner radius Rs1 may be greater than the planar antenna inner radius Rp1. However, it may be difficult to extend the planar antenna 210′.

Another assumption can be made. For example, it may be assumed that the inner radii of the antennas 210′ and 210 do not change. That is, it may be assumed that the three-dimensional antenna inner radius Rs1 is the same as the planar antenna inner radius Rp1. Assuming that the three-dimensional antenna inner radius Rs1 is the same as the planar antenna inner radius Rp1, the three-dimensional antenna outer radius Rs2 may be less than the planar antenna outer radius Rp2. It may be difficult to contract the planar antenna 210′. Therefore, in the process of converting the planar antenna 210′ into the three-dimensional antenna 210, corrugations may be formed on at least a portion of the three-dimensional antenna 210, or at least a portion of the three-dimensional antenna 210 may be folded and overlapped.

If the corrugations are formed on the three-dimensional antenna 210 or at least a portion of the three-dimensional antenna 210 is folded and overlapped, the performance of the three-dimensional antenna 210 may deteriorate, or it may be difficult to arrange the three-dimensional antenna 210 inside the lens body 100.

In particular, if irregular or excessive corrugations or folds occur in the three-dimensional antenna 210, the performance of the smart contact lens 10 may deteriorate, and the three-dimensional antenna 210 may protrude out of the lens body 100. Therefore, it is necessary to form a buffer section in the planar antenna 210′ to prevent the three-dimensional antenna 210 from being corrugated or folded and overlapped.

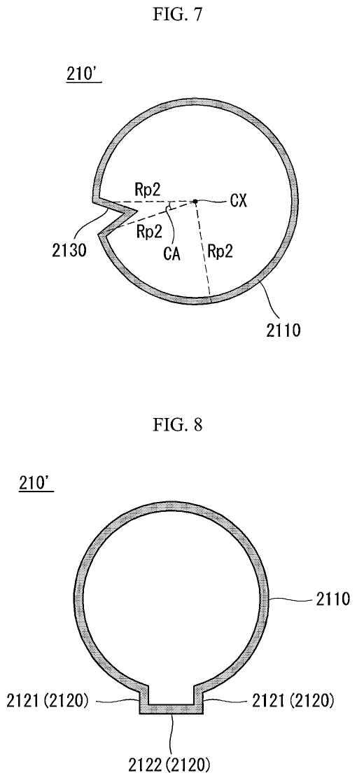

FIG. 7 illustrates a planar antenna provided with an antenna inner buffer.

Referring to FIGS. 6 and 7, there may be a need to form a buffer section in the planar antenna 210′ to prevent the three-dimensional antenna 210 from being corrugated or folded and overlapped. The buffer section may indicate a portion of the planar antenna 210′ that is bent or compressed due to a compression force generated in the process of converting the planar antenna 210′ into the three-dimensional antenna 210.

The planar antenna 210′ may include an antenna ring 2110. The antenna ring 2110 may form a ring shape. The planar antenna 210′ may include an antenna inner buffer 2130. The antenna inner buffer 2130 may indicate a portion of the antenna ring 2110 that is indented toward a center CX of the antenna ring 2110.

A compression angle CA may be a central angle of the antenna inner buffer 2130. That is, the compression angle CA may indicate an angle formed by the antenna inner buffer 2130 with respect to the center CX.

The compression force generated in the process of converting the planar antenna 210′ into the three-dimensional antenna 210 may be applied to the planar antenna 210′ in a circumferential direction of the antenna ring 2110. At a boundary between the antenna inner buffer 2130 and the antenna ring 2110, the antenna inner buffer 2130 may form an angle with the antenna ring 2110.

When the compression force is applied to the planar antenna 210′ in the circumferential direction of the antenna ring 2110, the antenna inner buffer 2130 may be contracted by the compression force based on the arrangement of the antenna inner buffer 2130 and the antenna ring 2110.

As the antenna inner buffer 2130 is contracted, contraction of the antenna ring 2110 can be suppressed or alleviated. A contractible distance of the antenna inner buffer 2130 may be a distance between both ends of the antenna inner buffer 2130.

The contractible distance of the antenna inner buffer 2130 may be greater than a difference between an outer perimeter of the planar antenna 210′ and an outer perimeter of the three-dimensional antenna 210. This can be expressed as shown in Equation 1 below.

$$Rp 2 \times CA \geq 2 π ( Rp 2 - Rs 2 ) \qquad [ Equation 1 ]$$

$$CA \geq \frac{2 π ( Rp 2 - Rs 2 )}{Rp 2} [ rad ]$$

If an angle formed between the planar antenna 210′ and the three-dimensional antenna 210 is denoted by “DA”, a difference between the planar antenna outer radius Rp2 and the three-dimensional antenna outer radius Rs2 may be a value by multiplying cos (DA) by the antenna width w. That is, the Equation 1 may be same as Equation 2 below.

$$CA \geq \frac{2 π \times w \times ( 1 - cos DA )}{Rp 2} [ rad ] \qquad [ Equation 2 ]$$

In FIGS. 6 and 7, the planar antenna inner radius Rp1 may be an inner radius of the antenna ring 2110 of the planar antenna 210′; the planar antenna outer radius Rp2 may be an outer radius of the antenna ring 2110 of the planar antenna 210′; the three-dimensional antenna inner radius Rs1 may be an inner radius of the antenna ring 2110 of the three-dimensional antenna 210; and the three-dimensional antenna outer radius Rs2 may be an outer radius of the antenna ring 2110 of the three-dimensional antenna 210.

FIG. 8 illustrates a planar antenna provided with an antenna outer buffer.

Referring to FIG. 8, the planar antenna 210′ may include an antenna outer buffer 2120. The antenna outer buffer 2120 may be formed to extend from the antenna ring 2110 toward the outside of the antenna ring 2110.

The antenna outer buffer 2120 may include an antenna outer buffer connector 2121 that is bent and extended outward from the antenna ring 2110. A plurality of antenna outer buffer connectors 2121 may be provided. For example, the antenna outer buffer connectors 2121 may include a pair of antenna outer buffer connectors 2121.

The antenna outer buffer 2120 may include an antenna outer buffer stand 2122. The antenna outer buffer stand 2122 may connect the pair of antenna outer buffer connectors 2121.

The antenna outer buffer 2120 may be adjacent to at least one of the sensor 220 (see FIGS. 1 and 2) and the chip 230 (see FIGS. 1 and 2). For example, the sensor 220 (see FIGS. 1 and 2) may be positioned adjacent to the antenna outer buffer 2120 and inside the planar antenna 210′. The chip 230 (see FIGS. 1 and 2) may be positioned inside the planar antenna 210′.

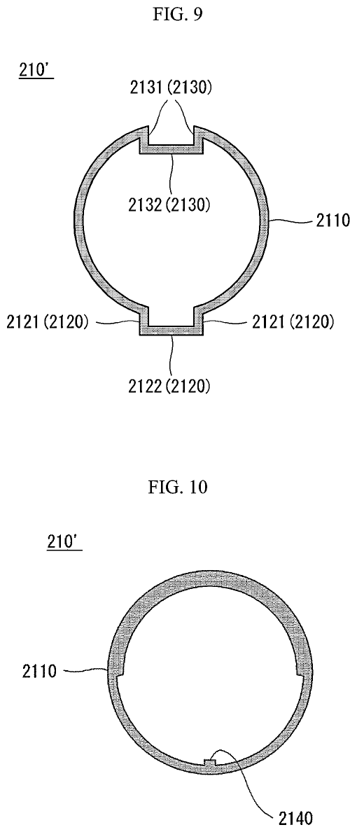

FIG. 9 illustrates a planar antenna provided with an antenna outer buffer and an antenna inner buffer.

Referring to FIG. 9, the planar antenna 210′ may include an antenna inner buffer 2130 and an antenna outer buffer 2120. The antenna outer buffer 2120 illustrated in FIG. 9 may be the same as the antenna outer buffer 2120 illustrated in FIG. 8. The antenna buffers 2120 and 2130 may indicate at least one of the antenna outer buffer 2120 and the antenna inner buffer 2130. The antenna buffers 2120 and 2130 may be formed by bending and extending from the antenna ring 2110 in a diameter direction of the antenna ring 2110.

The antenna inner buffer 2130 may include an antenna inner buffer connector 2131. The antenna inner buffer connector 2131 may be bent and extend inward from the antenna ring 2110. A plurality of antenna inner buffer connectors 2131 may be provided. For example, the antenna inner buffer connectors 2131 may include a pair of antenna inner buffer connectors 2131. The antenna buffer connectors 2121 and 2131 may indicate at least one of the antenna outer buffer connector 2121 and the antenna inner buffer connector 2131.

The antenna inner buffer 2130 may include an antenna inner buffer stand 2132. The antenna inner buffer stand 2132 may connect the pair of antenna inner buffer connectors 2131. The antenna buffer stands 2122 and 2132 may indicate at least one of the antenna outer buffer stand 2122 and the antenna inner buffer stand 2132.

The antenna inner buffer 2130 may be positioned opposite the antenna outer buffer 2120. The antenna outer buffer 2120 may accommodate at least one of the sensor 220 (see FIGS. 1 and 2) and the chip 230 (see FIG. 1). In this case, even if the compression force is applied to the antenna outer buffer 2120, contraction of the antenna outer buffer 2120 can be suppressed. That is, the degree to which the antenna inner buffer 2130 is contracted may be greater than the degree to which the antenna outer buffer 2120 is contracted.

FIGS. 10 to 17 illustrate a planar antenna according to various embodiments of the present disclosure.

Referring to FIG. 10, the antenna ring 2110 of the planar antenna 210′ may have different widths depending on a section. For example, a width of the antenna ring 2110 in one section of the antenna ring 2110 may be greater than a width of the antenna ring 2110 in another section of the antenna ring 2110.

The planar antenna 210′ may include an antenna inner protrusion 2140. The antenna inner protrusion 2140 may protrude toward the inside of the planar antenna 210′ at a specific portion of the antenna ring 2110. At least one of the sensor 220 (see FIGS. 1 and 2) and the chip 230 (see FIG. 1) may be connected to the antenna inner protrusion 2140.

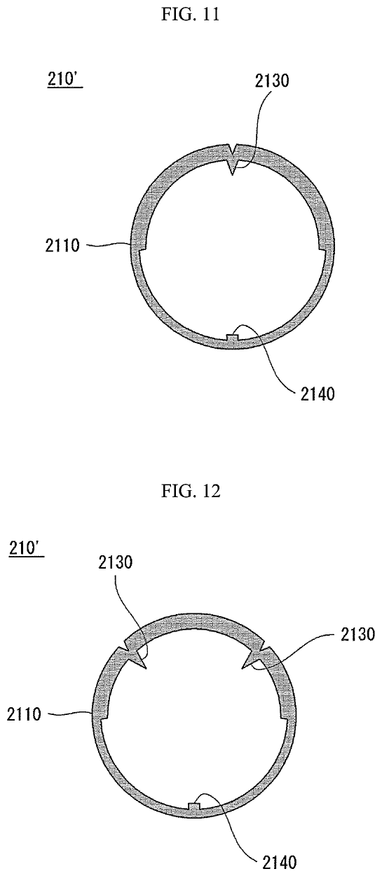

Referring to FIG. 11, the planar antenna 210′ may include an antenna inner protrusion 2140 and an antenna inner buffer 2130. The antenna inner buffer 2130 may be positioned opposite the antenna inner protrusion 2140.

The antenna inner buffer 2130 may form a recessed shape on an outer peripheral surface of the antenna ring 2110. For example, the antenna inner buffer 2130 may be recessed on the outer peripheral surface of the antenna ring 2110 to form a notch shape. The antenna inner buffer 2130 may form a shape that protrudes from an inner peripheral surface of the antenna ring 2110 toward the center of the planar antenna 210′.

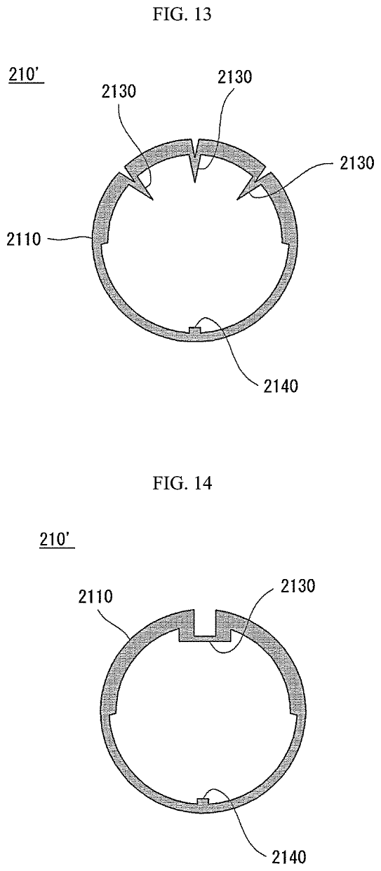

Referring to FIGS. 12 and 13, the planar antenna 210′ may include an antenna inner protrusion 2140 and an antenna inner buffer 2130. A plurality of antenna inner buffers 2130 may be provided. For example, two or three antenna internal buffers 2130 may be provided. The plurality of antenna inner buffers 2130 may be spaced apart from each other. The plurality of antenna inner buffers 2130 may be positioned opposite the antenna inner protrusion 2140.

Referring to FIGS. 11 to 13, a distance between both ends of the antenna inner buffer 2130 illustrated in FIG. 12 may be less than a distance between both ends of the antenna inner buffer 2130 illustrated in FIG. 11. There may be two antenna inner buffers 2130 illustrated in FIG. 12, and there may be one antenna inner buffer 2130 illustrated in FIG. 11. A distance between both ends of the antenna inner buffer 2130 illustrated in FIG. 13 may be less than the distance between both ends of the antenna inner buffer 2130 illustrated in FIG. 12. There may be three antenna inner buffers 2130 illustrated in FIG. 13, and there may be two antenna inner buffers 2130 illustrated in FIG. 12.

In other words, while the number of antenna inner buffers 2130 increases, the distance between both ends of the antenna inner buffer 2130 decreases. Hence, a contraction distance of the antenna inner buffer 2130 can be maintained constant. If the number of antenna inner buffers 2130 increases, the compression force applied to the planar antenna 210′ can be evenly distributed to the plurality of antenna inner buffers 2130.

Referring to FIG. 14, the planar antenna 210′ may include an antenna inner protrusion 2140 and an antenna inner buffer 2130. The antenna inner buffer 2130 may be positioned opposite the antenna inner protrusion 2140.

The antenna inner buffer 2130 may form a recessed shape on the outer peripheral surface of the antenna ring 2110. For example, the antenna inner buffer 2130 may be recessed on the outer peripheral surface of the antenna ring 2110 and have a shape of the antenna inner buffer 2130 illustrated in FIG. 9. For example, the antenna inner buffer 2130 may include an antenna inner buffer connector 2131 (see FIG. 9) and an antenna inner buffer stand 2132 (see FIG. 9).

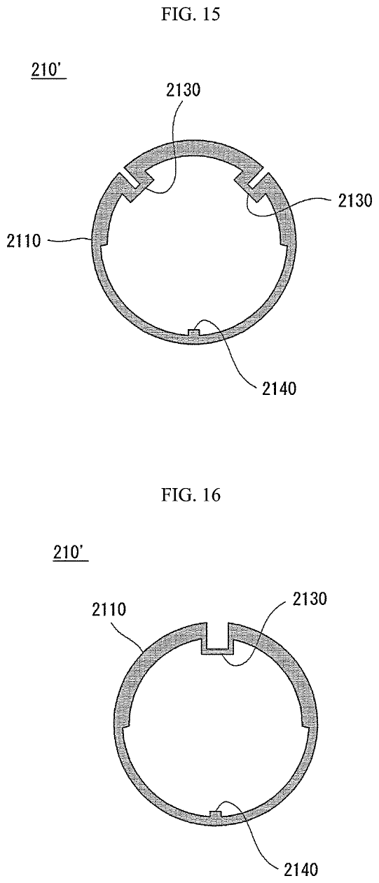

Referring to FIG. 15, the planar antenna 210′ may include an antenna inner protrusion 2140 and an antenna inner buffer 2130. A plurality of antenna inner buffers 2130 may be provided. For example, the antenna inner buffers 2130 may include a pair of antenna inner buffers 2130.

A distance between both ends of the antenna inner buffer 2130 illustrated in FIG. 15 may be less than a distance between both ends of the antenna inner buffer 2130 illustrated in FIG. 14. There may be two antenna inner buffers 2130 illustrated in FIG. 15, and there may be one antenna inner buffer 2130 illustrated in FIG. 14.

Referring to FIG. 16, a width of the antenna inner buffer connector 2131 (see FIG. 9) of the antenna inner buffer 2130 illustrated in FIG. 16 may be less than a width of the antenna inner buffer connector 2131 (see FIG. 9) of the antenna inner buffer 2130 illustrated in FIG. 14. As the width of the antenna inner buffer connector 2131 (see FIG. 9) decreases, it may be easy to contract the antenna inner buffer 2130.

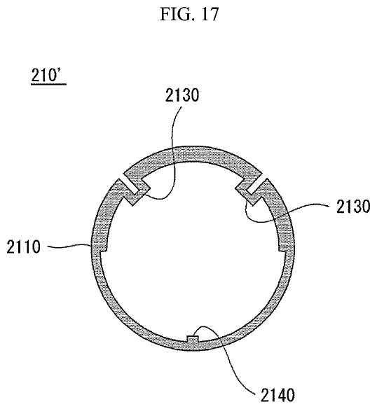

Referring to FIG. 17, there may be two antenna inner buffers 2130 illustrated in FIG. 17, and there may be one antenna inner buffer 2130 illustrated in FIG. 16. A distance between both ends of the antenna inner buffer 2130 illustrated in FIG. 17 may be less than a distance between both ends of the antenna inner buffer 2130 illustrated in FIG. 16.

Some embodiments or other embodiments of the present disclosure described above are not mutually exclusive or distinct from each other. Configurations or functions of some embodiments or other embodiments of the present disclosure described above can be used together or combined with each other.

It is apparent to those skilled in the art that the present disclosure can be embodied in other specific forms without departing from the spirit and essential features of the present disclosure. Accordingly, the above detailed description should not be construed as limiting in all aspects and should be considered as illustrative. The scope of the present disclosure should be determined by rational interpretation of the appended claims, and all modifications within an equivalent scope of the present disclosure are included in the scope of the present disclosure.

1. A smart contact lens comprising:
a lens body including a first lens surface in contact with an eye and a second lens surface positioned opposite the first lens surface; and
an antenna disposed between the first lens surface and the second lens surface and configured to form a closed loop,
wherein the antenna includes:
an antenna ring forming a shape of a ring as a whole; and
an antenna buffer bent and extended from the antenna ring in a diameter direction of the antenna ring.

2. The smart contact lens of claim 1, wherein the antenna buffer includes at least one of:
an antenna outer buffer bent and extended from the antenna ring toward an outside of the antenna ring in the diameter direction of the antenna ring; and
an antenna inner buffer bent and extended from the antenna ring toward an inside of the antenna ring in the diameter direction of the antenna ring.

3. The smart contact lens of claim 1, wherein the antenna buffer includes:
an antenna buffer connector bent and extended in the diameter direction of the antenna ring; and
an antenna buffer stand connected to the antenna buffer connector.

4. The smart contact lens of claim 3, wherein the antenna buffer connector includes a pair of antenna buffer connectors, and
wherein the antenna buffer stand connects the pair of antenna buffer connectors.

5. The smart contact lens of claim 1, wherein the antenna is formed by applying a pressure to a planar antenna formed on a plane and forms an angle with the planar antenna, and
wherein the planar antenna includes the antenna ring and the antenna buffer.

6. The smart contact lens of claim 5, wherein in a process of forming the antenna by applying the pressure to the planar antenna, a compression force is applied to the planar antenna, and
wherein, based on the compression force being applied to the planar antenna, the antenna buffer of the planar antenna is contracted.

7. The smart contact lens of claim 5, wherein a contractible distance of the antenna buffer of the planar antenna is greater than a difference between an outer perimeter of the planar antenna and an outer perimeter of the antenna.

8. The smart contact lens of claim 5, wherein a central angle of the antenna buffer of the planar antenna satisfies Equation 1:
$$CA \geq \frac{2 π ( Rp 2 - Rs 2 )}{Rp 2} [ rad ] \qquad [ Equation 1 ]$$
where Rp2 is an outer radius of the antenna ring of the planar antenna, Rs2 is an outer radius of the antenna ring of the antenna, and CA is the central angle of the antenna buffer of the planar antenna.

9. The smart contact lens of claim 5, wherein a central angle of the antenna buffer of the planar antenna satisfies Equation 2:
$$CA \geq \frac{2 π \times w \times ( 1 - cos DA )}{Rp 2} [ rad ] \qquad [ Equation 2 ]$$
where Rp2 is an outer radius of the antenna ring of the planar antenna, CA is the central angle of the antenna buffer of the planar antenna, DA is an angle between the planar antenna and the antenna, and w is a width of the planar antenna.

10. The smart contact lens of claim 1, further comprising:
a sensor and a chip disposed between the first lens surface and the second lens surface and connected to the antenna.

11. The smart contact lens of claim 10, wherein the antenna receives a power from an outside and transfers the power to the chip.

12. The smart contact lens of claim 10, wherein the sensor includes a strain sensor.
